(19) United States
(12) Patent Application Publication
(10) Pub. No.: US 2006/0088516 A1
(43) Pub. Date: Apr. 27, 2006

(54) Synthetic peptide combinations and methods of producing same

(75) Inventor: Gunther H. Klett-Loch, Mannheim (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Caroline Klett-Loch

(21) Appl. No.: 11/296,159

(22) Filed: Dec. 7, 2005

Related U.S. Application Data
(63) Continuation of application No. PCT/DE04/01229, filed on Jun. 17, 2004.

(30) Foreign Application Priority Data
Jun. 17, 2003 (DE) 103 27 518.5

(51) Int. Cl. A61K 38/43

(52) U.S. Cl. 424/94.1

(57) ABSTRACT

Preparation containing synthetic peptide combinations for application to humans or animals, wherein the peptide combinations originate from the partial hydrolysis of tissues from the human organism, wherein the peptide combinations are obtained from the neosynthesis of peptides, with the latter corresponding in their structure and composition to the tissue of the human organism, or wherein the peptide combinations are obtainable from genetically modified microorganisms, with the produced peptides corresponding with respect to their genetic starting material to the nucleotide sequences of peptides of the human organism. Also disclosed are methods for producing the synthetic peptide combinations.

### CROSS REFERENCE TO RELATED APPLICATION

The present application is a continuation of international application PCT/DE2004/0001229, filed 17 Jun., 2004, and which designates the U.S. The disclosure of the referenced application is incorporated herein by reference.

### FIELD OF THE INVENTION

The present invention relates to preparations that contain synthetic peptide combinations and are applied to humans and animals, and to methods for their production.

### BACKGROUND OF THE INVENTION

From scientific and medical studies, it becomes obvious that low-molecular proteins are able to intervene in a regulative manner in synthesis processes of the organism. This may lead to an improvement of the functionality of the tissues or endocrine system. Likewise, these low-molecular proteins, also known as signaling peptides, function as neurotransmitters on a neuronal level, or as differentiation factors on an immunological level. They are present in all tissues. Thus, for example, polypeptides regulate in the same way as interleukins and the GM-CSF (granulocyte macrophage colony stabilization factor), the early T-cell differentiation in the thymus epithelium. The synthetic reproduction of such and other polypeptides and their use in preparations make it thus possible to respond to an acquired immunodeficiency, infections, allergies, auto-immune diseases, neuroendocrine diseases (for example, carcinoid of the hyphophysis), as well as to immunorestorative effects, for example, after chemotherapy or radiation.

For example, WO 98/53845 discloses a synthetic, statistic thymic peptide combination and its use as an immunologically and/or endocrinologically active preparation, wherein synthetic thymic peptides corresponding to the thymus of the calf and their combination are employed for use in an immunologically and/or endocrinologically active preparation. The use of the synthetic, thymic peptide combinations in the preparation leads to a dosage-dependent increase in the proliferation of human lymphocytes. As a result, this preparation is used as medication in the treatment of immune weaknesses and immunodeficiencies, as well as in the treatment of hormonal disorders. Furthermore, this preparation is used as a substance for building up skin, hair, nails and as a cosmetic, as well as a food supplement. The synthetic thymic peptides originate in this case from a neosynthesis of peptides.

Quite in general, the known synthetic thymic peptides and their combination have the disadvantage that they correspond to the peptides and the peptide combination of the calf thymus, and that on the other hand only the thymus organ serves as template for the peptides and their combination. Thus, the use of these synthetic peptides and their combination in a preparation is limited to their described hormonal effect and the resultant synergistic endocrinological effect. In this respect, a treatment of special genetic, cell or tissue defects in organs or special tissue of humans with this preparation is not possible. Thus, for example, it would not be possible to use the signaling peptides that are effective for the thymus tissue, for treatment of functional disorders of the heart, liver, lung, or other organs, which result from genetic, cell or tissue defects. Likewise, functional disorders of the secondary lymph tissue, such as the lymph nodes, Peyer's plaques, intestine-associated lymphatic tissues, and spleen, remain totally uninfluenced. A further problem with the effective treatment in humans could arise in that the peptides that are isolated from the thymus of the calf differ from those of the thymus of humans, so that intolerance reactions to the animal protein result in some instances.

It is therefore an object of the invention to improve and further develop preparations of the initially described type, which contain synthetic peptide combinations, in such a manner that in addition to the treatment of hormonal and endocrinologial disorders, they can also be used in other fields of medical treatment, such as, for example, the treatment of genetic, cell and tissue defects. Furthermore, it is an object of the present invention to develop products with improved, specific effectiveness for humans on the basis of such peptide combinations. Likewise, it is an object to provide a method for producing a corresponding preparation with corresponding properties.

### SUMMARY OF THE INVENTION

The above and other objects and advantages of the invention are achieved, as regards the substance, by a preparation comprising a combination of synthetic peptides that are obtainable by treating tissues originating from a human organism with an enzyme combination that partially hydrolyzes the tissues. Accordingly, a preparation containing synthetic peptide combinations is characterized in that the peptide combinations originate from a partial hydrolysis of tissues, with the tissues originating from the human organism, and the partial hydrolysates being obtainable by means of special enzyme combinations.

### DETAILED DESCRIPTION OF THE INVENTION

In accordance with the invention it has been found that it is possible to obtain synthetic peptide combinations by the partial hydrolysis of human tissue by means of special enzyme combinations, which catalyze the hydrolysis under certain test conditions. These synthetic peptide combinations can be used as medication both for hormonal and endocrinological disorders and for dysfunctions of all tissues. Furthermore, it has been found that the use of synthetic peptides of the human organism makes it possible to achieve an improved effectiveness of the preparation. Intolerance and rejection reactions to animal proteins can be effectively avoided.

Concretely, it is provided that the synthetic peptide combinations can be obtained by means of special enzyme combinations. The enzyme combinations could be selected such that special, short-chain proteins from the tissues are partially hydrolyzed. This partial hydrolysis would thus permit isolating signaling peptides or other effective proteins, which could then be used in a preparation. Certain test conditions with respect to the pH-value, temperature, salt content, or duration of the hydrolysis in combination with the special enzyme combinations would enable a purposeful control of the partial hydrolysis.

In the case of the purposeful partial hydrolysis it would be advantageous, if it permitted obtaining peptides of a mass from 0.15 to 3 kd. This would ensure that a preparation can be administered by all ways of application.

The preparation containing the synthetic peptide combinations of the invention accomplishes the above-described object also by a preparation comprising neosynthetized peptides that correspond in their structure and composition to peptides and peptide combinations of tissues from a human organism. Accordingly, the preparation containing the synthetic peptide combinations is characterized in that the peptide combinations can be composed of neosynthetized peptides. In their structure and composition, the neosynthetized peptides correspond to peptides and peptide combinations of the tissue of the human organism.

In this respect, it has been found by the invention that the neosynthesis of peptides, which correspond to the peptides of the human organism, permit realizing peptide combinations, which can be used as medication for hormonal and endocrinological disorders as well as for dysfunctions of all tissues. It has furthermore been found that the de novo synthesis results in the possibility of being able to produce peptide and peptide combinations in a reproducible manner. As a result, it is possible to do without the use of explants of the human organism.

To produce these synthetic peptide combinations, the stepwise lineup of individual, chemically suitably derivatized amino acids to form short-chain peptides and the release of these synthetic peptides from their n-terminal group and derivatized form on side functions present themselves. With that, it is possible to produce these combinations in a pharmaceutically reproducible sterile manner.

The thus-produced peptide combinations have the advantage that unlike a natural preparation, they are able to eliminate risks of diseases, such as a prion risk, in particular BSE risk, or viral pathogens.

In an advantageous manner, the peptides could comprise an average mass from 0.15 to 3 kd. This would permit an easy de novo synthesis of these short-chain peptides within the scope of the tolerance range that applies to medications. Such a preparation could also be administered by all ways of application.

The preparation containing synthetic peptide combinations of the invention accomplishes the above-described object—within the scope of a further alternative—by a combination of synthetic peptide that are obtainable from genetically modified microorganisms, and wherein the genetic starting material of the synthetic peptide combinations correspond to nucleotide sequences of peptides of tissues from a human organism. Accordingly, the preparation containing the synthetic peptide combinations is characterized in that the peptide combinations can be obtained from genetically modified microorganisms. In this instance, the produced peptides correspond with respect to their genetic starting material to the nucleotide sequences of the peptides of tissues from the human organism.

Within the scope of this alternative realization, it has been found by the invention that cloning of the microorganisms with the genetic starting material that corresponds to the nucleotide sequences of the peptides of tissues from the human organism, permits producing synthetic peptides, which can be used as medication for hormonal and endocrinological disorders as well as for dysfunctions of all tissues. It has also been found that the use of microorganisms enables a pharmaceutical reproducibility on a large scale. In this respect, production could occur on an industrial scale in fluid cultures in fermenters, should there be a great demand for the preparations. In addition, the thus-produced peptides would be better tolerated by humans than comparable products from the animal organism.

An especially simple production of the synthetic peptides would be ensured by cloning cDNA in the microorganisms by means of plasmids. In this process, the cDNA could be obtained by means of a reverse transcription of the mRNA of the peptides. The use of cDNA in plasmids with possible selection markers on or in the selective medium would ensure that only those microorganisms are attracted, which contain the cDNA of the desired peptide. Besides the selection markers, the plasmids could also be provided with promoters, which effect an increased expression of the cloned cDNA.

In a very particularly advantageous manner, the microorganisms would be able to release the peptides into the culture medium, from which same could be collected. This could be realized by means of osmotic shocks or electric shock methods. It would thus be possible to obtain the synthetic peptides in a simple manner with subsequent centrifugation steps and chromatographic methods.

In a furthermore advantageous manner, the peptides could have an average mass from 0.15 to 3 kd. This enables a simple and stable cloning of the microorganisms, since the size of the nucleotide sequence is limited. Furthermore, the thus-produced peptide combinations can be administered in a preparation by all ways of application.

In a furthermore advantageous manner, the preparation containing the synthetic peptide combinations could be employed for use as medication in the treatment of organic dysfunctions. In this connection, it would be possible that defective or destroyed tissue is regenerated or newly built up by the effect of the synthetic peptide combinations.

It would also be possible to provide the preparation containing the synthetic peptide combinations as an immunologically and/or endocrinologically active preparation. In this connection, it is advantageous that the synthetic peptide combinations replicated from the human organ totally exclude a BSE risk or other diseases that can be transmitted from animals to humans.

It would also be possible to use the preparation containing the synthetic peptide combinations as cosmetic or food supplement. This would ensure that the preparation containing the synthetic peptide combinations would also be accessible to the user without medical indication.

As regards the method, the above-described object is accomplished by the step of treating tissue originating from a human organism with a combination of enzymes that partially hydrolyzes the tissue to produce a combination of synthetic peptides. Accordingly, a method for producing a preparation is characterized in that the tissue is partially hydrolyzed by means of enzymes, with the tissues originating from the human organism, and the partial hydrolysates being obtained by means of special enzyme combinations. Preferably, the method of the invention is used to produce a preparation wherein the enzyme combination is selected so that the partial hydrolysis of the tissue is controllable. The method can also be used to form a combination of synthetic peptides that have a mass from 0.15 to 3 kd. The preparation can be used as medication in the treatment of organic dysfunctions, treatment of immune weaknesses and immunodeficiencies, treatment of genetic defects, treatment of cell or tissue defects, treatment of hormonal disorders, as a cosmetic, and/or as food supplement. The foregoing part of the specification is herewith incorporated by reference for purposes of avoiding repetitions.

As regards the method, the above-described object is likewise accomplished by the step of lining up individual, chemically suitable derivatized amino acids to form neosynthesized peptides, wherein the formed neosynthetized peptides correspond in their structure and composition to peptides and peptide combinations of tissues from a human organism. Accordingly, a method for producing a preparation is characterized in that individual, chemically suitable derivatized amino acids are lined up to peptides, with the neosynthetized peptides corresponding in their structure and composition to peptides and peptide combinations of tissues of the human organism. In this connection, it would be possible to lay out the method in accordance with the Merrifield solid-phase method. Preferably, the method of the invention is used to produce a preparation wherein the synthetic peptides are obtainable from combining individual amino acids. The method can also be used to prepare a preparation containing synthetic peptide combinations wherein the synthetic peptides have an average mass from 0.15 to 3 kd. Additionally, the preparation containing synthetic peptide combinations can exclude excludes prions. The preparation can be used as medication in the treatment of organic dysfunctions, treatment of immune weaknesses and immunodeficiencies, treatment of genetic defects, treatment of cell or tissue defects, treatment of hormonal disorders, as a cosmetic, and/or as food supplement., so that also in this instance the foregoing part of the specification is herewith incorporated by reference for purposes of avoiding repetitions.

It is likewise possible to define a method of the invention—within the scope of a further alternative—by the step genetically modifying a microorganism to release a synthetic peptide into a culture medium; collecting the released synthetic peptide; and isolating the released synthetic peptide. Accordingly, a method for producing a preparation is characterized in that the peptides and peptide combinations are obtainable from genetically modified microorganisms. In this instance, the peptides correspond with respect to their genetic starting material to the nucleotide sequences of the peptides of tissues from the human organism. Preferably, the method of the invention is used to produce a preparation wherein the microorganisms are obtainable by cloning with cDNA sequences contained in plasmids. The method can also be used to prepare a preparation containing synthetic peptide combinations, wherein the microorganisms release synthetic peptides into a culture medium. In addition, the method can also be used to prepare a preparation containing synthetic peptide wherein the released synthetic peptides can be collected from the culture medium, and/or wherein the synthetic peptides that are released into the culture medium have an average mass from 0.15 to 3 kd. The preparation can be used as medication in the treatment of organic dysfunctions, treatment of immune weaknesses, treatment of immunodeficiencies, treatment of genetic defects, treatment of cell or tissue defects, treatment of hormonal disorders, as a cosmetic, and/or as food supplement. Likewise in this instance, the foregoing part of the specification is herewith incorporated by reference for purposes of avoiding repetitions.

Preferably, the method for producing a preparation could be developed to the extent that the peptide combinations obtained from partially hydrolyzed tissue, or the peptide combinations obtained from the neosynthesis of peptides, or the peptides and peptide combinations produced from genetically modified microorganisms form a quantity ratio and pattern of amino acids characteristic of the tissues of the human organism. In this way, it would be possible to ensure that nature-identical products result from these methods.

Finally, it should be remarked that there are countless possibilities of improving and further developing the teaching of the present invention in an advantageous manner. While the above-described embodiments serve to explain merely the claimed teaching, they do not limit it to the embodiments.

That which is claimed:

1. A preparation for application to humans and animals, the preparation comprising a combination of synthetic peptides that are obtainable by treating tissues originating from a human organism with an enzyme combination that partially hydrolyzes the tissues.

2. The preparation according to claim 1, wherein the enzyme combination is selected so that the partial hydrolysis of the tissue is controllable.

3. The preparation according to claim 2, wherein the the combination of synthetic peptides have a mass from 0.15 to 3 kd.

4. A preparation containing synthetic peptide combinations for application to humans and animals, wherein the peptide combinations comprises neosynthetized peptides, the neosynthetized peptides corresponding in their structure and composition to peptides and peptide combinations of tissues from a human organism.

5. The preparation containing synthetic peptide combinations according to claim 4, wherein the synthetic peptides are obtainable from combining individual amino acids.

6. The preparation containing synthetic peptide combinations according to claim 4, wherein the synthetic peptides have an average mass from 0.15 to 3 kd.

7. The preparation containing synthetic peptide combinations according to one of claim 4 wherein the combination of synthetic peptides excludes prions.

8. A preparation containing synthetic peptide combinations for application to humans and animals, wherein the peptide combinations are obtainable from genetically modified microorganisms, and wherein the genetic starting material of the synthetic peptide combinations correspond to nucleotide sequences of peptides of tissues from a human organism.

9. The preparation containing synthetic peptide combinations according to claim 8, wherein the microorganisms are obtainable by cloning with cDNA sequences contained in plasmids.

10. The preparation containing synthetic peptide combinations according to claim 8, wherein the microorganisms release synthetic peptides into a culture medium.

11. The preparation containing synthetic peptide combinations according to one of claim 10, wherein the released synthetic peptides can be collected from the culture medium.

12. The preparation containing synthetic peptide combinations according to one of claim 10, wherein the synthetic peptides that are released into the culture medium have an average mass from 0.15 to 3 kd.

13. The preparation according to claim 1, wherein the preparation is used in the treatment of organic dysfunctions, treatment of immune weaknesses, treatment of immunodeficiencies, treatment of genetic defects, treatment of cell or tissue defects, treatment of hormonal disorders, as a cosmetic, or as food supplement.

14. The preparation containing synthetic peptide combinations according to claim 4, wherein the preparation is used in the treatment of organic dysfunctions, treatment of immune weaknesses, treatment of immunodeficiencies, treatment of genetic defects, treatment of cell or tissue defects, treatment of hormonal disorders, as a cosmetic, or as food supplement.

15. The preparation containing synthetic peptide combinations according to claim 8, wherein the preparation is used in the treatment of organic dysfunctions, treatment of immune weaknesses, treatment of immunodeficiencies, treatment of genetic defects, treatment of cell or tissue defects, treatment of hormonal disorders, as a cosmetic, or as food supplement.

16. A method for producing a preparation according to claim 1, comprising:
treating tissue originating from a human organism with a combination of enzymes that partially hydrolyzes the tissue to produce a combination of synthetic peptides.

17. The method according to claim 16, further comprising the step of forming a weighted quantity ratio and a pattern of amino acids characteristic of tissues of a human organism.

18. A method of producing a preparation according to claim 4, comprising:
lining up individual, chemically suitable derivatized amino acids to form neosynthesized peptides, wherein the formed neosynthetized peptides correspond in their structure and composition to peptides and peptide combinations of tissues from a human organism.

19. The method for producing a preparation according to claim 18, further comprising the step of forming a weighted quantity ratio and a pattern of amino acids characteristic of tissues of a human organism.

20. A method for producing a preparation according to claim 8, comprising:
genetically modifying a microorganism to release a synthetic peptide into a culture medium;
collecting the released synthetic peptide; and
isolating the released synthetic peptide.

21. The method for producing a preparation according to claim 20, further comprising the step of forming a weighted quantity ratio and a pattern of amino acids characteristic of tissues of a human organism.
